(19) United States
(12) Patent Application Publication
(10) Pub. No.: US 2005/0256473 A1
(43) Pub. Date: Nov. 17, 2005

(54) Absorbent articles containing absorbent leg regions

(75) Inventors: Joseph R. Feldkamp, Appleton, WI (US); Gregory K. Hall, Menasha, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/835,525

(22) Filed: Apr. 29, 2004

(51) Int. Cl. A61F 13/15; A61F 13/20

(52) U.S. Cl. 604/378; 604/358; 604/385.01; 604/385.23

(57) ABSTRACT

Absorbent articles having leg extensions are disclosed. The absorbent articles may include, for instance, an outer cover, a liner, and an absorbent structure positioned in between the liner and the outer cover. The absorbent structure includes a crotch area and leg extensions that extend downwardly from the crotch area and are configured to lay adjacent the thighs of a user. The leg extensions assist in wicking liquids away from the crotch area when the absorbent article is insulted with a liquid. In one embodiment, the leg extensions comprise at least 30% by weight of the absorbent structure or, alternatively comprise at least 30% of the total surface area of the absorbent structure.

### BACKGROUND OF THE INVENTION

Many types of disposable consumer products such as diapers, training pants, feminine care articles, incontinence articles, and the like, utilize an absorbent pad structure for absorbing and wicking away body fluids. The absorbent structures are conventionally formed from an absorbent pad or batt, typically a fibrous material. For instance, the absorbent structures may be formed by employing conventional airlaying techniques wherein fibers and possibly a superabsorbent material are mixed and entrained in an airstream and then directed onto a forming surface to form the web.

Existing disposable absorbent articles typically have an absorbent structure that passes between the legs of a user at the position of the crotch, so as to cover the genitals, and then wrap upward toward the abdomen in the front and the buttocks in the rear. The absorbent structures are positioned in the crotch area in order to facilitate urine or body fluid capture. The portion of the absorbent structure at the vicinity of the crotch is usually rather thick and bulky in order to provide sufficient capacity for liquid storage. Ideally, liquid captured in the crotch portion of the absorbent structure migrates toward the remaining portions of the absorbent structure, namely towards the front portion and the rear portion. Migration of the liquid generally occurs by wicking. Since the front portion and the rear portion of the absorbent structure are positioned vertically upwards from the crotch area, however, wicking occurs against gravity and may be slow. Ultimately, fluids may accumulate in the crotch region of the absorbent structure creating a potential for leakage.

In view of the above, attempts have been made in the past to design an absorbent garment with increased total absorbent capacity and leak resistance. For instance, in U.S. patent application Publication No. 2003/0229329 A1 to Mercier, an absorbent garment is disclosed containing an absorbing material that includes a front section, a rear section, and a pair of opposing side sections. The opposing side sections extend partially and to a very limited extent down the leg of a user when the garment is being worn. In particular, the side sections primarily provide extra absorption near the leg/hip crease and inner thigh leg length areas when the garment is attached to a user, and also to mitigate against gasket failure.

The inner side sections of the garment shown in Mercier, however, are relatively small in comparison to the entire size of the fluid absorbent material. Because the opposing side sections are positioned downwardly from the crotch area only to a limited extent, the side sections have a tendency to accumulate excessive amounts of fluid due to gravity-assisted wicking of fluids from the crotch area into the inner side sections. In this regard, the present inventors have discovered that relatively small side sections as shown in Mercier may easily become saturated with fluid during use of the garment. As the side sections become saturated with fluid, the inner thigh of a user becomes wet which creates many of the same problems that the side sections are intended to alleviate, such asleakage.

In view of the above, a need currently exists for an improved absorbent article that includes absorbent areas that extend downwardly along the inner thighs of a user. In particular, a need exists for an absorbent article in which the downwardly extending absorbent areas surrounding the inner thighs are enlarged in order to effectively absorb liquids without becoming saturated with fluids.

### SUMMARY OF THE INVENTION

The present invention is generally directed to a liquid absorbent article, such as a diaper, training pants, feminine hygiene product, adult incontinence product, swim undergarment, and the like.

In one embodiment, the liquid absorbent article includes an outer cover and an inner liner formed into the shape of a garment. The garment includes a front region and a back region that forms a waist opening therebetween. The garment further includes leg regions defining leg openings. The leg regions extend a determined distance downwardly along the legs of a user. For instance, purely for exemplary purposes, the leg regions may extend at least about one half inch, such as at least about one inch, or at least about two inches down the legs of a user. The distance the leg regions extend generally depends upon the particular application and the desired result. For instance, when producing absorbent articles for infants and small children, the leg regions may extend from about one-half inch to about four inches. When producing absorbent articles for use by adults, however, the leg regions may extend from about two inches to about ten inches, such as from about four inches to about eight inches.

The liquid absorbent article further includes an absorbent structure positioned in between the outer cover and the inner liner. The absorbent structure comprises a crotch area positioned between the front region and the back region of the garment. In accordance with the present invention, the crotch area is further in communication with a pair of downwardly extending leg areas. The leg areas are positioned within the leg regions of the garment and are configured to lay adjacent to and make contact with the inner thighs of a user. The leg regions containing the leg areas may also contact the outer thighs of a user depending upon the configuration.

The leg areas comprise a substantial portion of the absorbent structure. For instance, the leg areas may comprise at least about 30% by weight of the absorbent structure and/or may comprise at least about 30% of the total surface area of the absorbent structure.

More particularly, the leg areas may comprise at least about 40% by weight of the absorbent structure, such as at least about 50% by weight of the absorbent structure and, in one embodiment, may comprise from about 35% by weight to about 85% by weight of the absorbent structure. Similarly, the leg areas may also comprise at least about 40% of the total surface area of the absorbent structure, such as at least about 50% of the total surface area of the absorbent structure, and, in one embodiment, may comprise from about 35% to about 85% of the total surface area of the absorbent structure.

In general, the leg areas of the absorbent structure should have sufficient liquid holding capacity so that the leg areas do not become saturated with liquids when the absorbent garment is insulted with a body fluid. For instance, the capacities of the leg areas should be sufficient to hold or accommodate at least about 30% of the expected liquid loading. For instance, when producing absorbent articles for infants and small children, the leg areas should be capable of holding from about 30 grams to about 450 grams of liquid without becoming saturated. When the absorbent articles are to be worn by adults, the leg areas should be capable of absorbing from about 60 grams to about 1000 grams of liquid without becoming saturated. For feminine hygiene products, the leg areas should be capable of holding from about 3 grams to about 25 grams of a menstrual fluid without becoming saturated. It should be understood that each of the above numerical ranges are provided for exemplary purposes. It should also be understood that each of the above values may be viewed as a minimum and that the leg areas may have a greater capacity than any of the values listed above.

The downwardly extending leg areas may be integral with the crotch area of the absorbent structure or, alternatively, may be comprised of separate pieces that are attached to the crotch area. The downwardly extending leg areas may also partially encircle or fully encircle the legs of a user when the garment is being worn. For instance, the leg areas may curve around the leg regions of the garment at least about 150 degrees, such as at least about 180 degrees, and in one embodiment, may curve around the leg regions at least about 210 degrees.

The crotch area of the absorbent structure may include a front portion that is positioned in the front region of the garment and a middle portion. In addition, the crotch area can also include a back portion that is positioned in the back region of the garment.

The absorbent structure may be formed from various absorbent materials. For instance, the absorbent structure may be formed from pulp fibers, superabsorbent particles, and mixture thereof. The absorbent structure may also have a single layer construction or a multi-layer construction. For instance, in one embodiment, at least portions of the absorbent structure may be made from two layers of absorbent material. The first layer, which is configured to lay adjacent to the inner liner or surge layer, may have a density less than the density of a second layer which lays adjacent to the outer cover. In this manner, fluids will have a tendency to accumulate in the second layer away from the skin of a user.

The liquid absorbent article may contain various elastic members for providing the article with form-fitting properties. Leg gaskets may also be present on the article for preventing fluid leakage if necessary. The absorbent article may be in a one-piece construction that is pulled over the legs of a user. Alternatively, the front region of the garment may be releasably attached to the back region of the garment using any suitable attachment structure. For instance, in one embodiment, hook and loop fasteners may be provided for attaching the front region to the back region. In an alternative embodiment, a releasable adhesive may be used.

Other features and aspects of the present invention will be made apparent from the following detailed description.

### BRIEF DESCRIPTION OF THE FIGURES

A full and enabling disclosure of the present invention, including the best mode thereof to one of ordinary skill in the art, is set forth more particularly in the remainder of the specification, including reference to the accompanying figures, in which:

FIG. 1 is a perspective view of one embodiment of a liquid absorbent article made in accordance with the present invention;

FIG. 2 is another perspective view of the embodiment illustrated in FIG. 1;

FIG. 3 is a perspective view of an alternative embodiment of a liquid absorbent article made in accordance with the present invention;

FIG. 4 is a perspective view of another alternative embodiment of a liquid absorbent article made in accordance with the present invention;

FIG. 5A is a plan view of one embodiment of an absorbent structure that may be incorporated into a liquid absorbent article of the present invention;

FIG. 5B is a perspective view illustrating the absorbent structure shown in FIG. 5A incorporated into an absorbent article in accordance with the present invention;

FIG. 6A is a plan view of an alternative embodiment of an absorbent structure made in accordance with the present invention;

FIG. 6B is a perspective view of the absorbent structure illustrated in FIG. 6A;

FIG. 7 is a plan view of another alternative embodiment of an absorbent structure made in accordance with the present invention;

FIG. 8 is a top view illustrating the absorbent structure of FIG. 7;

FIG. 9 is a plan view of still another embodiment of an absorbent structure made in accordance with the present invention;

FIG. 10 is a plan view of another alternative embodiment of an absorbent structure made in accordance with the present invention;

FIG. 11 is a plan view of still another alternative embodiment of an absorbent structure made in accordance with the present invention; and

FIGS. 12 and 13 are plan views of absorbent structures illustrating the results obtained in the Example described below.

Repeat use of reference characters in the present specification and drawings is intended to indicate the same or analogous features or elements of the invention.

### DETAILED DESCRIPTION OF THE INVENTION

It is to be understood by one of ordinary skill in the art that the present discussion is a description of exemplary embodiments only, and is not intended as limiting the broader aspects of the present invention.

In general, the present invention is directed to the construction of disposable absorbent articles. Such articles include, for instance, but are not limited to diapers, children's training pants, feminine care articles (such as pantiliners, pads and interlabial products), incontinence articles, swim pants, and the like.

In accordance with the present invention, the absorbent articles not only include a front region and a back region that passes between the legs of a user, but also include leg regions that extend a determined distance downwardly along the legs of a user. In comparison to conventional diapers and other similar garments, the absorbent articles of the present invention have a design similar to “biker-style” shorts worn by athletes, with leg regions that cover an appreciable portion of the thighs. In order to absorb liquids, the absorbent article includes an absorbent structure that includes a crotch area and leg areas that are positioned within the leg regions of the garment. In this manner, absorbent material extends downwardly from a crotch area to be positioned adjacent the legs of a user.

Many benefits and advantages are obtained when incorporating into an absorbent article an absorbent structure that includes downwardly extending leg areas. For instance, by including leg areas made from an absorbent material, fluids that contact the crotch area of the absorbent structure are wicked away from the crotch area and gravity drain to the leg areas for more uniform distribution of liquid waste. The leg areas of the garment also form a natural gasket for preventing fluid leaks.

In one embodiment, the amount of absorbent material used to form the garment is comparable to conventional articles except with the addition of greater surface area due to the downwardly extending leg areas. Thus, the absorbent structure may be somewhat thinner than conventional products allowing for the production of garments having an overall thin construction with improved fit and comfort. The absorbent articles made in accordance with the present invention are well suited for active children and adults with an absence of material bunching in the sensitive crotch region and, since the garments may have a stream-lined shape with form-fitting properties, a reduction of abrasion since the garments are not loosely worn about the wearer.

As described above, the absorbent structure incorporated into the absorbent article of the present invention includes downwardly extending leg areas that assist in capturing fluids and wicking away fluids from the crotch area where the fluids normally are deposited. Since the leg areas are in a downward position from the crotch area, wicking of fluids from the crotch area is gravity assisted. The present inventors have discovered that apparently due to the forces of gravity, substantial amounts of fluid accumulate in the leg areas. Thus, in accordance with the present invention, the leg areas comprise a substantial portion of the overall weight and/or surface area of the absorbent structure.

In particular, in accordance with the present invention, the leg areas for most applications comprise at least about 30% by weight of the absorbent structure and/or at least about 30% of the total surface area of the absorbent structure. For instance, the leg areas may comprise at least about 40% or at least about 50% of the weight and/or surface area of the absorbent structure. In one particular embodiment, for instance, the leg areas may comprise from about 35% to about 80% of the total weight and/or of the total surface area of the absorbent structure. In this manner, the leg areas have sufficient liquid capacity to absorb fluids and still have a dry feeling to the user. Further, ensuring that the leg areas have sufficient absorbent capacity prevents against the formation of fluid leaks.

For example, in general, the leg areas should have sufficient liquid capacity to hold and accommodate at least 30% of expected liquid loading depending upon the type of product being produced. More particularly, the leg areas should have sufficient capacity to hold at least 35% of expected liquid loading, such as at least about 45% of expected liquid loading. When producing infant and child products, for instance, the leg areas may have a minimum liquid capacity of from about 30 grams to about 450 grams without becoming saturated. For adult incontinence products, the leg areas may have a liquid capacity of from about 60 grams to about 1000 grams before becoming saturated. For feminine hygiene products, on the other hand, the leg areas may have a capacity to hold menstrual fluids in an amount of from about 3 grams to about 25 grams without becoming saturated. It should be understood that greater capacities may be desired in certain applications.

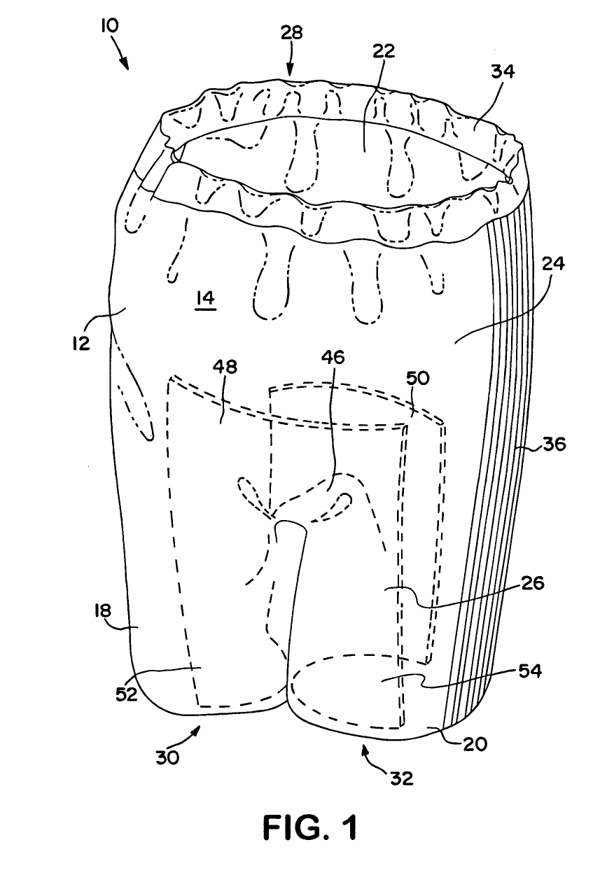

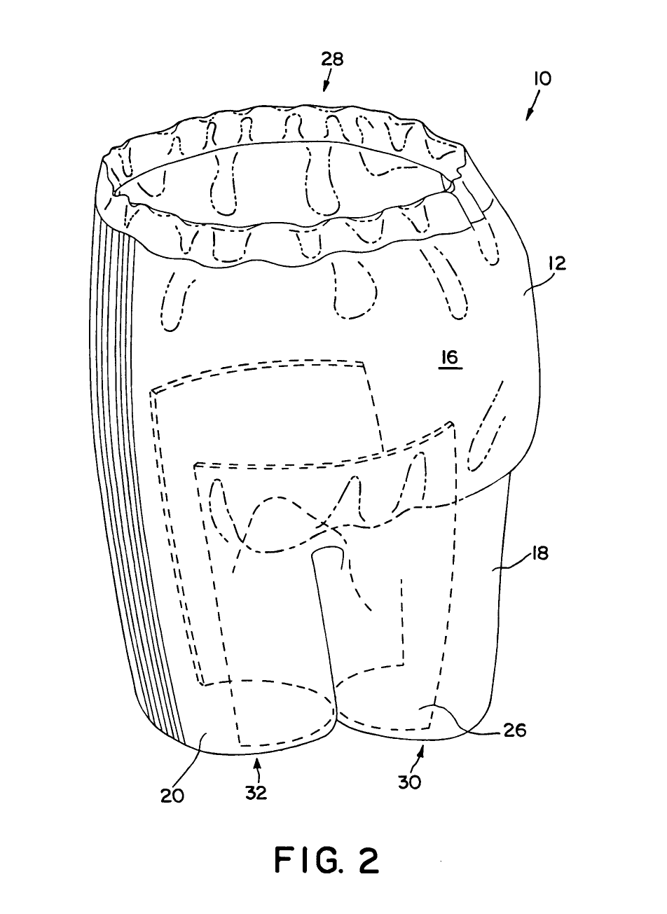

Referring to FIGS. 1 and 2, one embodiment of an absorbent article made in accordance with the present invention generally 10 is shown. For exemplary purposes only, the absorbent article 10 may be, for instance, a diaper, a training pant, a swim pant, or an adult incontinence article. The absorbent article 10 includes a chassis 12 defining a front region 14 and a back region 16 as shown in FIG. 2. In accordance with the present invention, the chassis 12 further includes a first leg region 18 and a second leg region 20.

The chassis 12 includes a bodyside liner 22 which is configured to contact the wearer, and an outer cover 24 positioned opposite the bodyside liner which is configured to contact the wearer's clothing. An absorbent structure 26 is positioned or located between the inner lining 22 and the outer cover 24.

As shown in FIGS. 1 and 2, the absorbent article 10 forms a garment that includes a waist opening 28 and a pair of leg openings 30 and 32.

To enhance containment and/or absorption of body exudates and in order to provide the absorbent article with form-fitting properties, the absorbent article 10 may include various elastic members positioned at different locations on the article. Alternatively, the entire outer cover and/or inner liner may be stretchable and/or elastic. In the embodiments shown in FIGS. 1 and 2, the absorbent article 10 includes a waist elastic member 34. The waist elastic member 34 can be operatively joined to the outer cover 24 and/or to the bodyside liner 22. The waist elastic member 34 may extend over the entire length of the waist opening 28 as shown in FIGS. 1 and 2 or only over a portion of the waist opening. Further, multiple waist elastic members may be incorporated into the article.

In addition to the waist elastic member 34, the absorbent article 10 shown in FIGS. 1 and 2 further includes at least one side elastic member 36. For instance, a single side elastic member may be positioned on one side of the article or a pair of side elastic members may be positioned on opposite sides of the article.

The waist elastic member 34 and the side elastic member 36 can be formed of any suitable elastic material. As is well known to those skilled in the art, suitable elastic materials include sheets, strips, strands or ribbons of natural rubber, synthetic rubber, or thermoplastic elastomeric polymers. The elastic materials can be stretched and attached to a substrate, attached to a gathered substrate, or attached to a substrate and then elasticized or shrunk, for example with the application of heat; such that elastic constrictive forces are imparted to the substrate. In one particular embodiment, for instance, one of the elastic members may include a plurality of dry-spun coalesced multi-filament spandex elastomeric threads sold under the tradename LYCRA and available from E.I. DuPont de Nemours and Co., Wilmington, Del.

In other embodiments, the elastic members may be made from a stretch-bonded laminate, a neck-bonded laminate, or made using any other suitable laminate material. The elastic members may be attached to the article 10 using any suitable attachment means. For instance, the elastic members may be attached using adhesive, thermal or ultrasonic bonding.

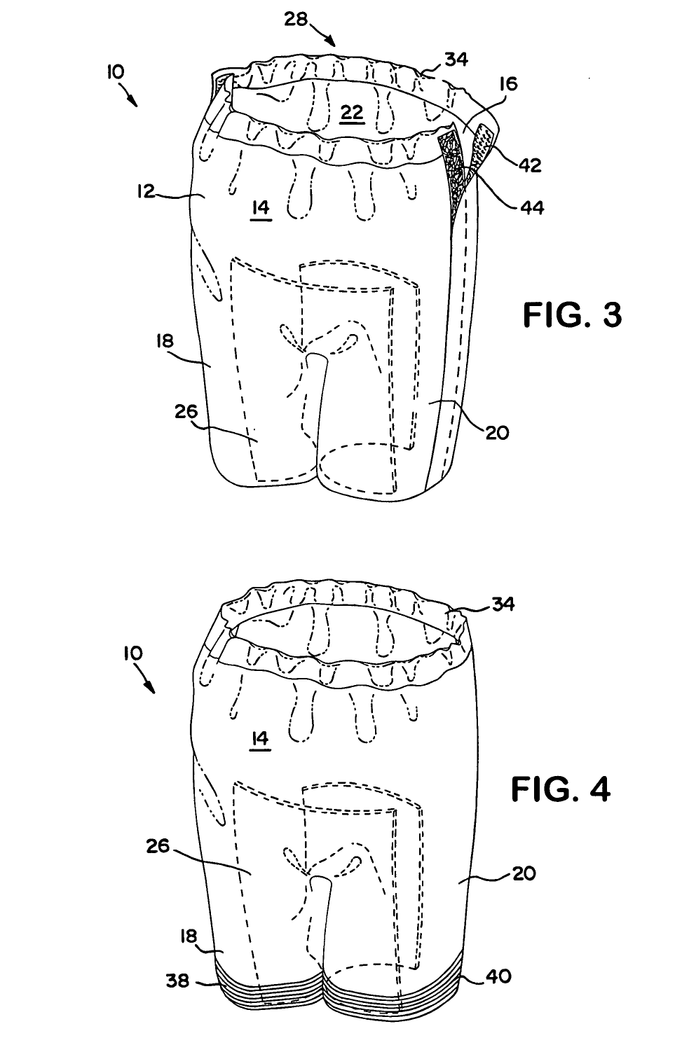

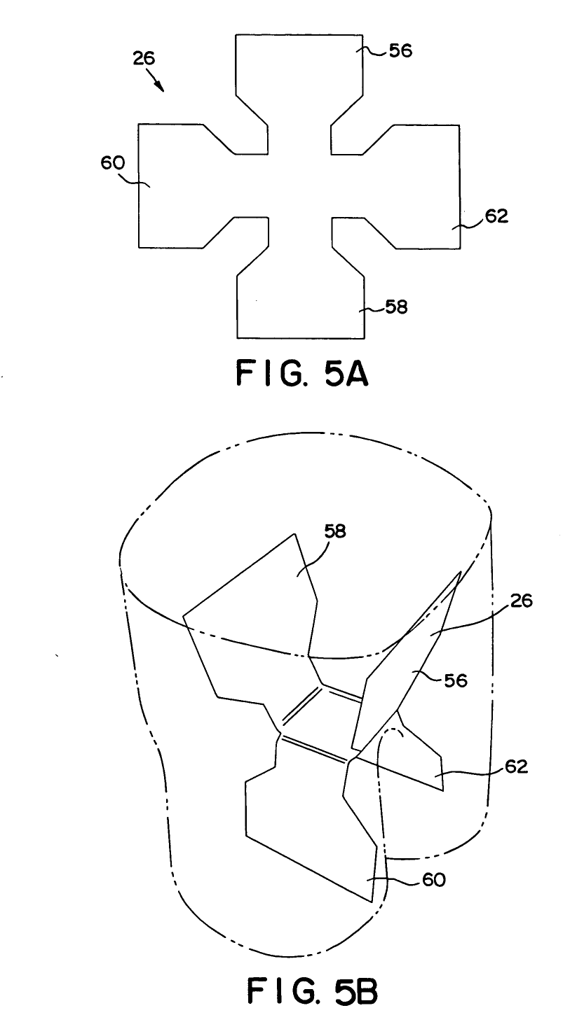

Referring to FIGS. 3 and 4, alternative embodiments of absorbent articles made in accordance with the present invention are shown. Like reference numerals have been used in order to illustrate similar elements. Referring to FIG. 4, an absorbent article 10 is shown that includes a first leg elastic member 38 and a second leg elastic member 40 positioned so as to define the terminal end of the leg openings. In this embodiment, the leg elastic members 38 and 40 not only provide form-fitting properties to the garment but also serve as gasket members that prevent fluid leakage. The elastic members 38 and 40 may be made from any of the materials described above. Further, it should be understood that the leg elastic members 38 and 40 may be used in conjunction with the side elastic member 36 shown in FIGS. 1 and 2 to form still another embodiment of the present invention.

In FIGS. 1, 2 and 4, absorbent articles are illustrated having a unitary construction. In FIG. 3, however, an absorbent article 10 is shown in which the front region 14 is releasably attached to the back region 16 using a suitable attachment structure. In general, any attachment structure may be used that is adapted to refastenably connect the front region and the back region together. In one particular embodiment as shown in FIG. 3, the attachment structure may include hook and loop type fasteners. For instance, as shown, the back region 16 includes a strip of hook material 42, while the front region 14 includes a corresponding strip of loop material 44. It should be understood, however, that in addition to hook and loop fasteners, various other attachment structures may be used. For instance, in an alternative embodiment, the attachment structure may comprise an adhesive positioned opposite a releasable surface.

In the embodiment shown in FIG. 3, the attachment structure extends the entire length of the absorbent article 10. In alternative embodiments, however, the attachment structure may only extend along a partial length of the absorbent article. For instance, the attachment structure may extend from the waist opening 28 to a position where the leg openings begin. In this embodiment, the article may first be pulled over the legs of a user and then the front region 14 may be attached to the back region 16.

In still another alternative embodiment, it should be understood that the hook material 42 and/or the loop material 44 may be attached to an elastic material which is then secured to the article. The elastic material may be any of the elastic materials described above.

As shown in FIGS. 1 through 4, the absorbent structure 26 is positioned within the absorbent article 10 in between the liner 22 and the outer cover 24. As shown particularly in FIG. 1, the absorbent structure 26 includes a crotch area 46 positioned between the front region 14 and the back region 16 of the garment. In the embodiment illustrated, the crotch area 46 includes a front area 48 positioned within the front region of the garment and a back area 50 positioned within the back region of the garment. In other embodiments, the back area 50 of the absorbent structure may be eliminated.

In accordance with the present invention, the absorbent structure 26 further includes a first leg area 52 and a second leg area 54. The leg areas 52 and 54 extend downwardly from the crotch area 46. The leg areas 52 and 54 are positioned so as to lay adjacent to the inner thighs of a user.

As shown in FIG. 1, the leg areas 52 and 54 comprise a significant portion of the absorbent structure 26. For instance, as described above, the leg areas 52 and 54 may comprise at least 30% by weight of the absorbent structure and/or may comprise at least 30% of the total surface area of the absorbent structure.

In some applications, the leg areas 52 and 54 of the absorbent structure 26 also surround a substantial portion of the leg openings 30 and 32. For example, the leg areas 52 and 54 may curve around the leg regions of the absorbent article by at least about 150°, such as at least about 180°. In one particular embodiment, for instance, the leg areas 52 and 54 may curve around the leg regions by at least about 210°.

The material used to form the absorbent structure 26 may vary depending upon the particular application. In general, any suitable liquid absorbent material may be used.

The material used to form the absorbent structures, for example, may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from US Alliance Pulp Mills of Coosa, Ala., USA, and is a bleached, highly absorbent wood pulp containing primarily soft wood fibers. As a general rule, the superabsorbent material is present in the absorbent web in an amount of from about 0 to about 90 weight percent based on total weight of the web. The web may have a density within the range of about 0.01 to about 0.5 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 10 times its weight in liquid, and suitably is capable of absorbing more than about 25 times its weight in liquid. Suitable superabsorbent materials are readily available from various suppliers. For example, FAVOR SXM 880 superabsorbent is available from Stockhausen, Inc., of Greensboro, N.C., USA; and Drytech 2035 is available from Dow Chemical Company, of Midland, Mich., USA.

In one embodiment, the absorbent structure may contain different types of superabsorbent materials in order to carefully control the absorbent properties of the product. For instance, in one embodiment, different superabsorbent materials may be used in order to regulate the flow of liquids into the leg absorbents. For example, superabsorbent materials having a larger particle size may be used in the crotch area 46 while smaller superabsorbent particles may be used in the leg areas 52 and 54. In this manner, the leg areas 52 and 54 may have a faster rate of absorption than the crotch area 46, causing liquids to move quickly into the leg areas where the liquids are stored. In this embodiment, for instance, the crotch area may contain superabsorbent particles having an average particle size of greater than about 400 microns while the leg areas may contain superabsorbent particles having an average particle size of less than about 400 microns.

In addition to cellulosic fibers and superabsorbent materials, the absorbent pad structures may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives can enhance the integrity of the bulk absorbent structure, and alternatively or additionally may provide adherence between facing layers of the folded structure.

The absorbent materials may be formed into a web structure by employing various conventional methods and techniques. For example, the absorbent web may be formed with a dry-forming technique, an airlaying technique, a carding technique, a meltblown or spunbond technique, a wet-forming technique, a foam-forming technique, or the like, as well as combinations thereof. Layered and/or laminated structures may also be suitable. Methods and apparatus for carrying out such techniques are well known in the art.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles or fibers, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. No. 4,100,324 to Anderson. et al.; U.S. Pat. No. 5,284,703 to Everhart, et al.; and U.S. Pat. No. 5,350,624 to Georger. et al.; which are incorporated herein in their entirety by reference for all purposes.

It is also contemplated that elastomeric absorbent web structures may be particularly well suited to the present invention. For example, an elastomeric coform absorbent structure having from about 35% to about 65% by weight of a wettable staple fiber, and greater than about 35% to about 65% by weight of an elastomeric thermoplastic fiber may be used to define absorbent pad structures according to the invention. Examples of such elastomeric coform materials are provided in U.S. Pat. No. 5,645,542, incorporated herein in its entirety for all purposes. As another example, a suitable absorbent elastic nonwoven material may include a matrix of thermoplastic elastomeric nonwoven filaments present in an amount of about 3 to less than about 20% by weight of the material, with the matrix including a plurality of absorbent fibers and a super-absorbent material each constituting about 20-77% by weight of the material. U.S. Pat. No. 6,362,389 describes such a nonwoven material and is incorporated herein by reference in its entirety for all purposes. Absorbent elastic nonwoven materials are useful in a wide variety of personal care articles where softness and conformability, as well as absorbency and elasticity, are important.

The web may have a density in the range of about 0.01 to about 0.5 grams per cubic centimeter. In a particular aspect of the invention, the absorbent web material can be provided with an absorbent capacity of at least about 8 g/g employing 0.9 wt % saline (8 grams of 0.9 wt % saline per gram of absorbent web). The absorbent capacity of the absorbent web can alternatively be at least about 9 g/g, and can optionally be at least about 15 g/g to provide improved benefits. Additionally, the absorbent capacity may be up to about 40 g/g, or more, to provide desired performance.

The absorbent material web is also selected so that the individual absorbent pad structures possess a particular individual total absorbency depending on the intended article of use. For example, for infant care products, the total absorbency is expected to be within the range of about 100-900 grams of 0.9 wt % saline, and can typically be about 500 g of 0.9 wt % saline. For adult care products, the total absorbency can be within the range of about 200-2000 grams of 0.9 wt % saline, and can typically be about 1300 g of saline. For feminine care products, the total absorbency can be within the range of about 7-50 grams of menstrual fluid or menses simulant, and can typically be within the range of about 30-40 g of menstrual fluid or menses simulant.

In one embodiment, the absorbent structure may be made from a layered absorbent material. The absorbent material may include, for instance, a first layer and a second layer. The first layer, which is to be positioned adjacent the liner 22, may have a lower density than the second or outer layer. The first layer, for example, may have a high contact angle, such as greater than about 40 degrees, while the second layer may have a higher density and may be prepared from fine, high wetting fibers. In this embodiment, liquids contacting the absorbent structure are quickly absorbed by the first layer and then transported and stored in the second layer. Such an arrangement may greatly reduce skin-liquid contact, keeping the skin relatively dry.

The absorbent structure 26 may also be embossed if desired. For instance, liquid channels may be embossed in the absorbent structure for directing fluids to a particular location. In one embodiment, for instance, the absorbent structure may be embossed with columns that extend from the crotch area 46 into the leg areas 52 and 54. The embossed channels may serve to direct fluids into the leg areas if desired.

The basis weight of the absorbent structure may vary dramatically depending upon the particular product being produced. In general, the basis weight of the absorbent structure may vary from about 50 gsm to about 800 gsm. The basis weight may be uniform or may vary depending upon the location. When the basis weight is substantially uniform, the leg areas 52 and 54 may have a tendency to account for a substantial portion of the surface area of the structure. In other embodiments, however, the basis weight of the absorbent structure may change based on location. For example, zoned absorbent structures may be used having relatively thick areas in combination with relatively thin areas. In this embodiment, for instance, the crotch area may have a thickness greater than the rest of the absorbent structure. In other embodiments, the leg areas may be relatively thick with or without a crotch area that is relatively thick.

In other embodiments, however, the basis weight and/or density of the absorbent structure 26 may vary depending upon location in order to not only efficiently absorb fluids at a particular location but also to modify the overall shape of the absorbent structure. For instance, in one embodiment, the bottom crotch area 46 may have a higher basis weight than the remainder of the absorbent structure. Further, the leg areas 52 and 54 may be made smaller by increasing the basis weight of the leg areas in relation to the remainder of the absorbent structure.

As described above, the absorbent structure 26 is positioned in between the liner 22 and the outer cover 24 as shown in FIG. 1. The liner and outer cover may be made from various materials.

The outer cover 24 may be made from a material that is substantially liquid impermeable, and can be elastic, stretchable or nonstretchable. The outer cover 24 can be a single layer of liquid impermeable material, or may include a multi-layered laminate structure in which at least one of the layers is liquid impermeable. For instance, the outer cover 24 can include a liquid permeable outer layer and a liquid impermeable inner layer that are suitably joined together by a laminate adhesive.

For example, in one embodiment, the liquid permeable outer layer may be a spunbond polypropylene nonwoven web. The spunbond web may have, for instance, a basis weight of from about 15 gsm to about 25 gsm.

The inner layer, on the other hand, can be both liquid and vapor impermeable, or can be liquid impermeable and vapor permeable. The inner layer is suitably manufactured from a thin plastic film, although other flexible liquid impermeable materials may also be used. The inner layer prevents waste material from wetting articles such as bedsheets and clothing, as well as the wearer and caregiver. A suitable liquid impermeable film may be a polyethylene film having a thickness of about 0.2 mm.

A suitable breathable material that may be used as the inner layer is a microporous polymer film or a nonwoven fabric that has been coated or otherwise treated to impart a desired level of liquid impermeability. Other “non-breathable” elastic films that may be used as the inner layer include films made from block copolymers, such as styrene-ethylene-butylene-styrene or styrene-isoprene-styrene block copolymers.

As described above, the absorbent structure is positioned in between the outer cover and a liquid permeable bodyside liner 22. The bodyside liner 22 is suitably compliant, soft feeling, and non-irritating to the wearer's skin. The bodyside liner 22 can be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner 22. For example, the bodyside liner can be made from a meltblown or spunbonded web of polyolefin fibers. The bodyside liner can also be a bonded-carded web composed of natural and/or synthetic fibers.

In one embodiment, the bodyside liner may be liquid permeable in the crotch area 46 but may be liquid impermeable in the leg regions 18 and 20. In this embodiment, the bodyside liner 22 may be made from different materials. Alternatively, a bodyside liner may be constructed from a single material that is selectively permeable. For instance, in one embodiment, the bodyside liner can be made from an impermeable film that is apertured at least in the crotch area.

A suitable liquid permeable bodyside liner 22 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular embodiment, the polypropylene forms the core and the polyethylene forms the sheath of the fiber. Other fiber orientations, however, are possible.

In addition to a liner and outer cover, the absorbent article may also include a surge layer positioned in between the liner and the absorbent structure. Surge layers are constructed to quickly collect and temporarily hold liquid surges, and to transport the temporarily held liquid to the absorbent structure.

Various woven and non-woven fabrics can be used to construct the surge layer. For example, the surge layer may be a layer made of a meltblown or spunbond web of synthetic fibers, such as polyolefin fibers. The surge layer may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge layer may be composed of a substantially hydrophobic material, and the hydrophobic material may optionally be treated with a surfactant or otherwise processed to impart a desired level of wettability and hydrophilicity.

Examples of materials suitable for the surge layer are set forth in U.S. Pat. No. 5,486,166 issued Jan. 23,1996 in the name of C. Ellis et al. and entitled “Fibrous Nonwoven Web Surge Layer For Personal Care Absorbent Articles And The Like”; U.S. Pat. No. 5,490,846 issued Feb. 13, 1996 in the name of Ellis et al. and entitled “Improved Surge Management Fibrous Nonwoven Web For Personal Care Absorbent Articles And The Like”; and U.S. Pat. No. 5,364,382 issued Nov. 15, 1994 in the name of Latimer et al. and entitled “Absorbent Structure Having Improved Fluid Surge Management And Product Incorporating Same”, the disclosures of which are hereby incorporated by reference in a manner consistent with the present document.

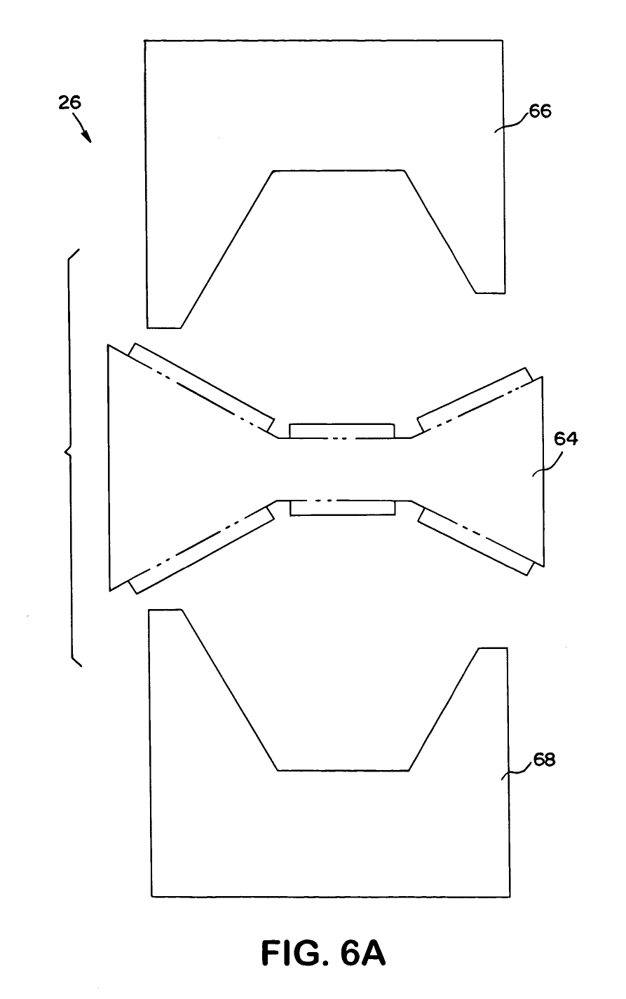

Referring to FIGS. 5A through 11, various different shapes of absorbent structures that may be used in absorbent articles made in accordance with the present invention are illustrated. Referring to FIGS. 5A and 5B, an absorbent structure 26 is shown that has the shape of a cross. In particular, the absorbent structure 26 includes four flaps 56, 58, 60 and 62. Referring to FIG. 5B, the absorbent structure 26 shown in 5A is illustrated contained within an absorbent garment. As shown, the flaps 58 and 56 are folded upwardly to form a front area and a back area, while the flaps 62 and 60 are folded downwardly to form leg areas. In this embodiment, assuming the absorbent structure is made from a uniform basis weight, the leg areas 60 and 62 comprise at least about 30 to 45% by weight of the absorbent structure.

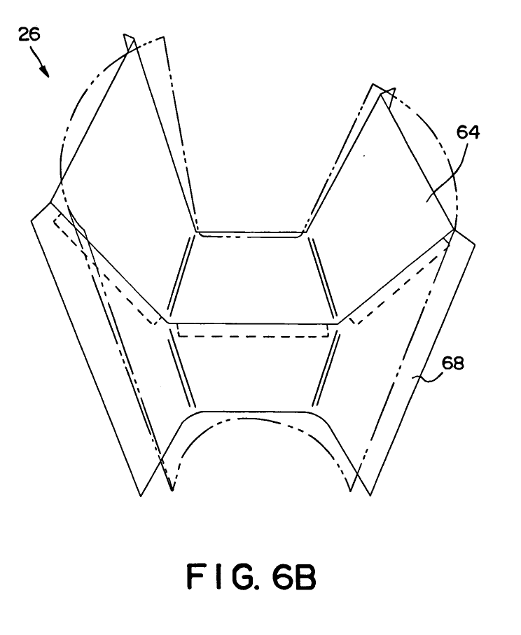

Referring to FIGS. 6A and 6B, another alternative embodiment of an absorbent structure 26 made in accordance with the present invention is shown. In this embodiment, the absorbent structure 26 is comprised of three sections, a middle section 64 and a pair of side sections 66 and 68. Middle section 64 as shown in FIG. 6B forms the crotch area including a front area and a back area. The side sections 66 and 68, on the other hand, form leg areas that extend downwardly from the crotch area. The sections 64, 66 and 68 may be attached together using any suitable attachment means, such as by using an adhesive. Alternatively, the absorbent structure 26 may be formed from a unitary piece.

In the embodiment shown in FIG. 6B, the leg areas account for from about 60% to about 70% of the surface area of the absorbent structure and, assuming that the absorbent structure is made from a uniform basis weight, also account for about 60% to about 70% by weight of the absorbent structure.

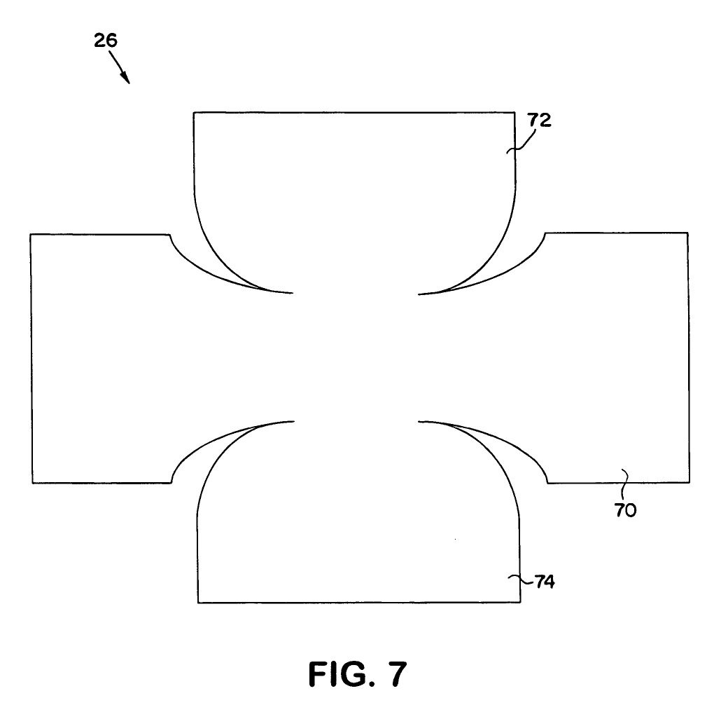

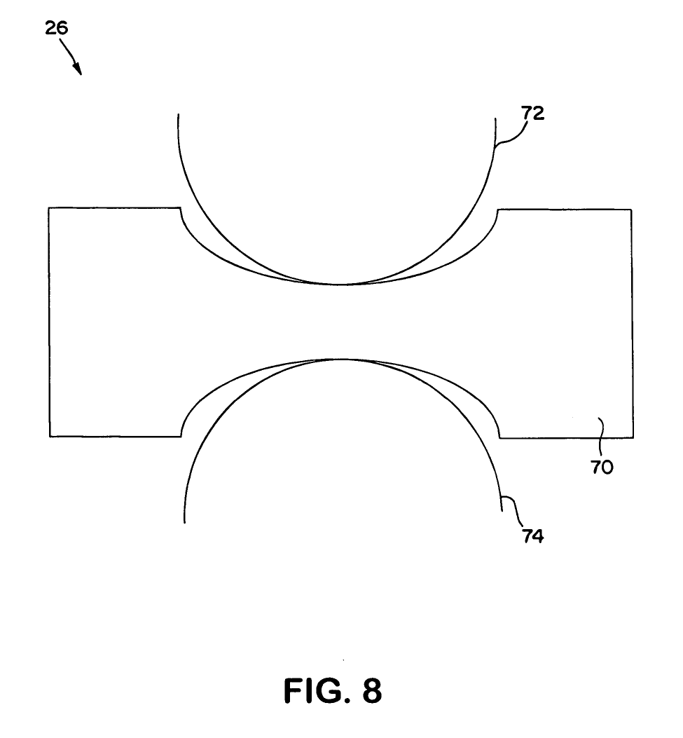

Still another embodiment of an absorbent structure generally 26 made in accordance with the present invention is shown in FIGS. 7 and 8. As shown, the absorbent structure includes a crotch area 70 in communication with a pair of leg areas 72 and 74. Referring to FIG. 8, a top plan view of the absorbent structure 26 as it would appear in an absorbent garment is illustrated. As shown, the leg areas 72 and 74 extend downwardly from the crotch area 70 and wrap at least partially around the legs of a user.

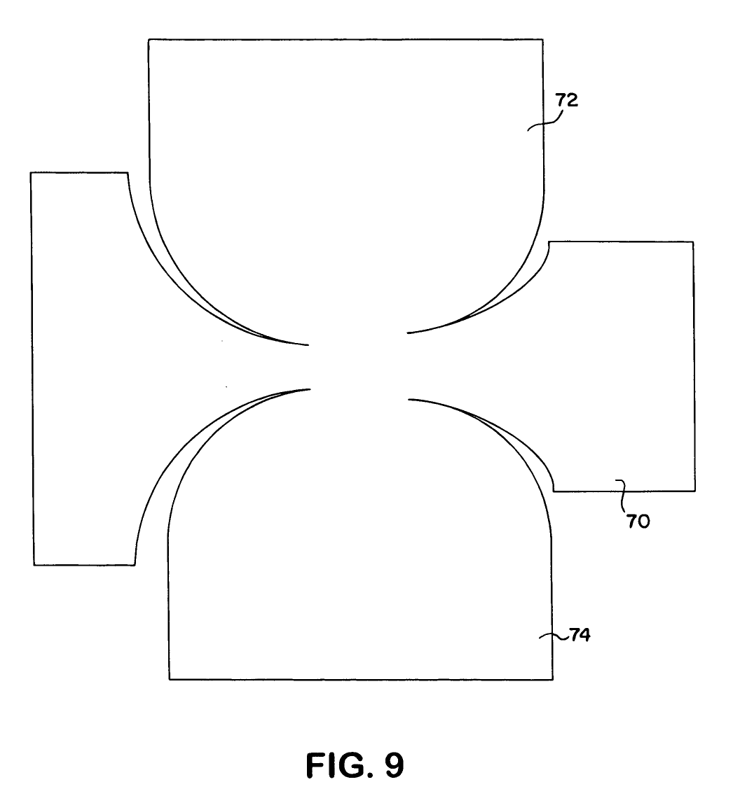

Referring to FIGS. 9 through 11, further examples of absorbent structures are illustrated in accordance with the present invention. The absorbent structures illustrated in FIGS. 9 through 11 are somewhat similar to the absorbent structure illustrated in FIG. 7. As such, like reference numerals have been used to indicate similar elements.

As shown in FIGS. 9 through 11, the size and shape of the crotch area 70 and the size and shape of the leg areas 72 and 74 are varied. The crotch area 70 and the leg areas 72 and 74 may be varied, for instance, in order to produce an absorbent structure having a shape well suited for incorporation into an absorbent article and/or a shape well suited for absorbing liquids for a particular application.

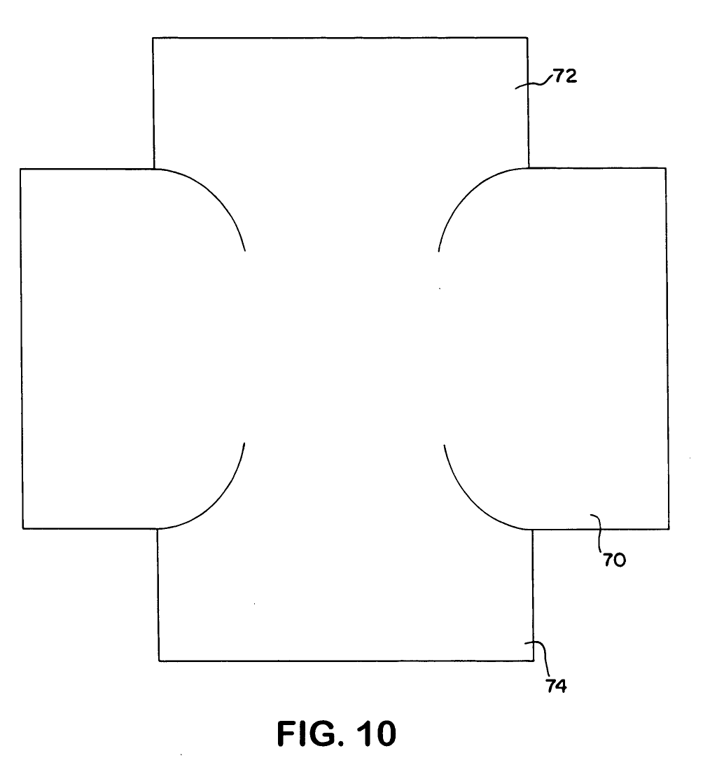

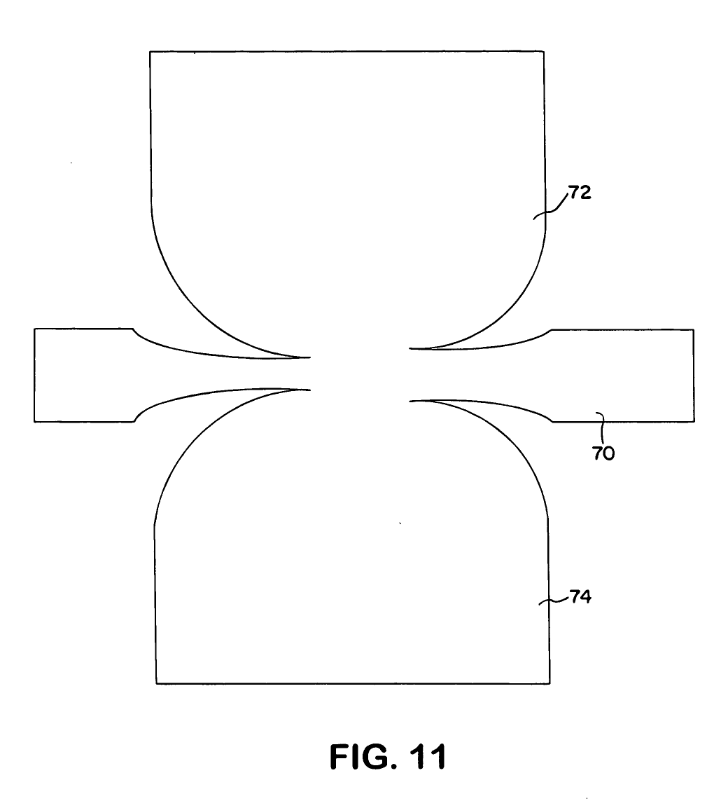

As shown in FIG. 9, the crotch area 70 includes a relatively thin middle section separated by relatively large leg areas 72 and 74. In FIG. 10, on the other hand, a middle section of the crotch area 70 is relatively large in comparison to the leg areas 72 and 74. In FIG. 11, the crotch area 70 includes a streamlined front area and a streamlined back area and is relatively thin in the middle.

It should be noted that in the designs illustrated, the leg areas comprise a substantial portion of the overall shape of the absorbent structure. As described above, the leg areas are to be configured so as to absorb significant amounts of liquid without becoming saturated during normal use. The liquid capacity of the leg areas may be varied by either varying mass and/or surface area.

The present invention may be better understood with reference to the following example.

### EXAMPLE

The following example is provided in order to demonstrate the advantages of the present invention, using finite element simulation computer software.

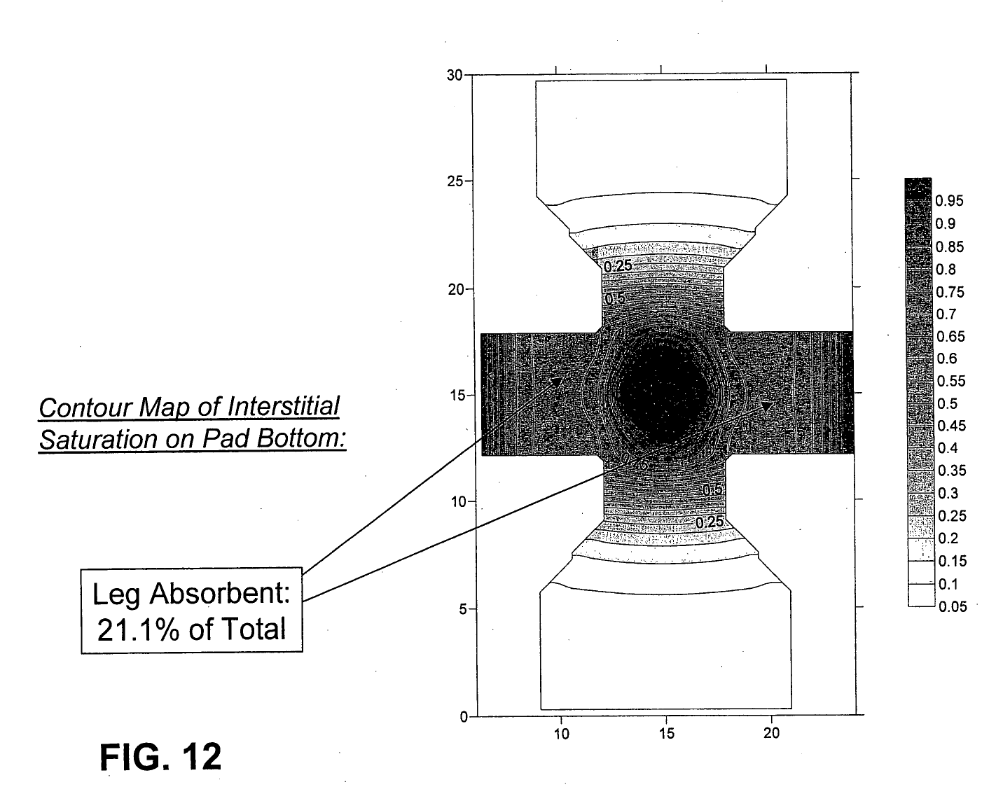

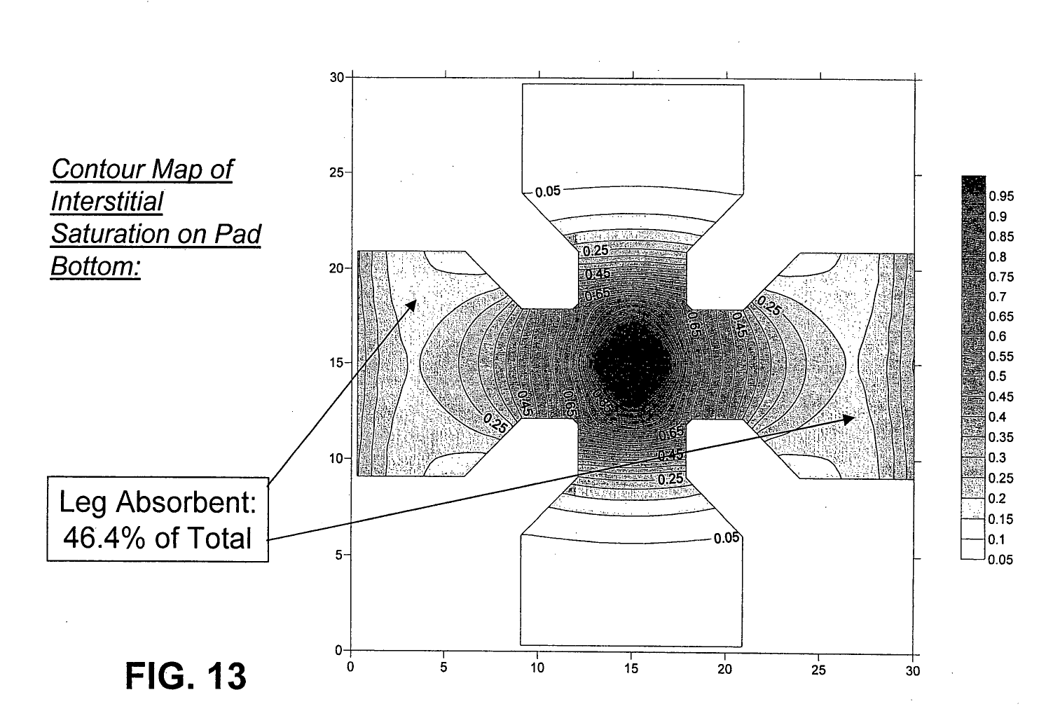

The software, which is capable of modeling absorbency of three-dimensional heterogeneous media, was used to simulate two two-layered absorbent structures, one with a cross shape as shown in FIG. 13 and one with a truncated cross shape as shown in FIG. 12. FIG. 13 depicts an absorbent structure made in accordance with the present invention, while FIG. 12 is provided for comparative reasons, showing relatively small leg regions.

Both absorbent structures were simulated in a position with the front and back flaps pointed upward and the two leg flaps pointed downward as depicted in FIG. 5B. As shown in FIG. 13, the leg portions of the cross-shaped absorbent structure are more than 30% of the total pad, and the leg portions of the truncated cross-shaped pad, shown in FIG. 12, are less than about 30% of the entire pad.

The absorbent structure shown in FIG. 12 and the absorbent structure shown in FIG. 13 were programmed into the computer software to be made from the same two-layer material. In particular, the absorbent structures included a first absorbent layer, that may be considered a surge layer, that had a density of 0.08 g/cc and a thickness of 0.2 cm under no load. The first layer contained liquid absorbent fibers having a diameter of 30 microns and had a contact angle of 60 degrees.

The second layer of each absorbent structure had a density of 0.2 g/cc and a thickness of 0.2 cm. The second layer contained liquid absorbent fibers having a diameter of 20 microns, contained 60% by weight of a superabsorbent material and had a contact angle of 10 degrees.

The program simulated insulting the two-layer absorbent structures with 30 grams of liquid in the diamond insult zone shown in the middle of the pads. The computer modeling was programmed such that the liquid comprised 30 grams of saline at room temperature and that uptake occurred in a time frame of about 40 seconds to about 50 seconds. In general, the computer software simulated migration of the liquid over time. The program also was programmed to assume that an external load was placed on the absorbent structure of 1.0 kPa.

The program produced gray-scale contour plots of the bottom horizon of the two-layer absorbent structures, where regions of high interstitial saturation are black to dark gray and regions of lower interstitial saturation are light gray. The results of the simulation are shown in FIGS. 12 and 13. In particular, the results illustrate the absorbent structures 46 seconds and 42 seconds respectively after being insulted with the liquid.

Referring to FIG. 12, for instance, liquid absorption of the truncated cross-shaped absorbent structure is shown. As illustrated, the leg regions collected substantial amounts of liquid. Specifically, the leg regions collected more liquid than the upper portions and reached saturations exceeding 95% at the lower edges.

FIG. 13, on the other hand, shows liquid absorbency of an absorbent structure made in accordance with the present invention. Again, the leg areas collected more liquid than the upper areas. In this configuration, however, the leg areas only reached about 35% saturation. Thus, the absorbent structure illustrated in FIG. 13 provided various advantages and benefits in view of the absorbent structure shown in FIG. 12. In particular, the absorbent structure shown in FIG. 13 would have a drier feeling after being insulted with the liquid than the structure shown in FIG. 12. The absorbent structure shown in FIG. 13 would also be better at preventing against leakage.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A liquid absorbent article comprising:
an outer cover and an inner liner, the article including a front region and a back region, the article further including leg regions, and wherein, when the article is positioned about a user, the leg regions extend a determined distance downwardly along the legs of a user; and
an absorbent structure positioned in between the outer cover and the inner liner, the absorbent structure comprising a crotch area positioned between the front region and the back region of the article, the crotch area being in communication with a pair of downwardly extending leg areas, the leg areas being positioned within the leg regions of the article and configured to lay adjacent to the inner thighs of a user, the leg areas comprising at least about 30% by weight of the absorbent structure.

2. A liquid absorbent article as defined in claim 1, wherein the leg areas comprise at least about 40% by weight of the absorbent structure.

3. A liquid absorbent article as defined in claim 1, wherein the leg areas comprise from about 35% to about 85% by weight of the absorbent structure.

4. A liquid absorbent article as defined in claim 1, wherein the leg areas comprise at least about 50% by weight of the absorbent structure.

5. A liquid absorbent article as defined in claim 1, wherein the leg areas extend a distance downwardly of at least about one half inch.

6. A liquid absorbent article as defined in claim 1, wherein the leg areas extend a distance downwardly of from about two inches to about eight inches.

7. A liquid absorbent article as defined in claim 1, wherein the leg areas extend a distance downwardly of at least about four inches.

8. A liquid absorbent article as defined in claim 1, wherein the leg regions include elastic members for creating form-fitting properties.

9. A liquid absorbent article as defined in claim 1, wherein the absorbent structure comprises pulp fibers.

10. A liquid absorbent article as defined in claim 1, wherein the absorbent structure comprises superabsorbent particles.

11. A liquid absorbent article as defined in claim 1, wherein the absorbent structure comprises pulp fibers and superabsorbent particles.

12. A liquid absorbent article as defined in claim 1, wherein the leg areas include a first layer and a second layer, the first layer being positioned against the inner liner while the second layer being positioned against the outer cover, the first layer having a lower density than the second layer.

13. A liquid absorbent article as defined in claim 1, wherein the crotch area comprises a front portion and a middle portion.

14. A liquid absorbent article as defined in claim 13, wherein the crotch area further comprises a back portion.

15. A liquid absorbent article as defined in claim 1, wherein the leg areas are integral with the crotch area of the absorbent structure.

16. A liquid absorbent article as defined in claim 1, wherein the front region and the back region of the article are releasably attached together by an attachment structure.

17. A liquid absorbent article as defined in claim 16, wherein the attachment structure comprises hook and loop fasteners.

18. A liquid absorbent article as defined in claim 1, wherein the article comprises a diaper.

19. A liquid absorbent article as defined in claim 1, wherein the article comprises a training pant.

20. A liquid absorbent article as defined in claim 1, wherein the article comprises an adult incontinence product.

21. A liquid absorbent article as defined in claim 1, wherein the leg areas curve around the leg regions by at least about 150 degrees.

22. A liquid absorbent article as defined in claim 1, wherein the leg areas curve around the leg regions by at least about 180 degrees.

23. A liquid absorbent article as defined in claim 1, wherein the leg areas curve around the leg regions by at least about 210 degrees.

24. A liquid absorbent article as defined in claim 1, wherein the inner liner is liquid impermeable in the leg regions.

25. A liquid absorbent article as defined in claim 1, wherein the inner liner is apertured in a crotch region and is liquid impermeable in the leg regions.

26. A liquid absorbent article as defined in claim 1, wherein the leg areas of the absorbent structure have sufficient capacity to hold at least 30% by weight of expected liquid loading.

27. A liquid absorbent article comprising:
an outer cover and an inner liner formed into the shape of a garment, the garment including a front region and a back region that form a waist opening therebetween, the garment further including leg regions defining leg openings, and wherein, when the garment is positioned about a user, the leg regions extend a determined distance downwardly along the legs of a user; and
an absorbent structure positioned in between the outer cover and the inner liner, the absorbent structure comprising a crotch area positioned between the front region and the back region of the garment, the crotch area being in communication with a pair of downwardly extending leg areas, the leg areas being positioned within the leg regions of the garment and configured to lay adjacent to the inner thighs of the user, the leg areas comprising at least about 30% of the surface area of the absorbent structure.

28. A liquid absorbent article as defined in claim 27, wherein the leg areas comprise at least about 40% of the surface area of the absorbent structure.

29. A liquid absorbent article as defined in claim 27, wherein the leg areas comprise from about 35% to about 85% of the surface area of the absorbent structure.

30. A liquid absorbent article as defined in claim 27, wherein the leg areas also comprise at least about 30% by weight of the absorbent structure.

31. A liquid absorbent article as defined in claim 27, wherein the leg areas also comprise at least about 40% by weight of the absorbent structure.

32. A liquid absorbent article as defined in claim 27, wherein the leg areas curve around the leg regions by at least about 150 degrees.

33. A liquid absorbent article as defined in claim 28, wherein the leg areas curve around the leg regions by at least about 180 degrees.

34. A liquid absorbent article as defined in claim 28, wherein the leg areas curve around the leg regions by at least about 210 degrees.

35. A liquid absorbent article as defined in claim 27, wherein the leg regions include elastic members for creating form-fitting properties.

36. A liquid absorbent article as defined in claim 27, wherein the absorbent structure comprises pulp fibers.

37. A liquid absorbent article as defined in claim 27, wherein the absorbent structure comprises superabsorbent particles.

38. A liquid absorbent article as defined in claim 27, wherein the absorbent structure comprises pulp fibers and superabsorbent particles.

39. A liquid absorbent article as defined in claim 27, wherein the leg areas include a first layer and a second layer, the first layer being positioned against the inner liner while the second layer being positioned against the outer cover, the first layer having a lower density than the second layer.

40. A liquid absorbent article as defined in claim 27, wherein the crotch area comprises a front portion and a middle portion.

41. A liquid absorbent article as defined in claim 27, wherein the leg areas are integral with the crotch area of the absorbent structure.

42. A liquid absorbent article as defined in claim 27, wherein the inner liner is liquid impermeable in the leg regions.

43. A liquid absorbent article comprising:
an outer cover and an inner liner formed into the shape of a garment, the garment including a front region and a back region that forms a waist opening therebetween, the garment further including leg regions defining leg openings and wherein the garment is positioned about a user, the leg regions extend a determined distance downwardly along the legs of a user, the determined distance being at least one half inch; and
an absorbent structure positioned in between the outer cover and the inner liner, the absorbent structure being made from a material comprising pulp fibers, superabsorbent particles, or mixtures thereof, the absorbent structure comprising a crotch area positioned between the front region and the back region of the garment, the crotch area including at least a front portion and a middle portion, the crotch area being in communication with a pair of downwardly extending leg areas, the leg areas being positioned within the leg regions of the garment and configured to lay adjacent to the inner thighs of a user, the leg areas comprising at least about 30% by weight of the absorbent structure and at least about 30% of the surface area of the absorbent structure, the leg areas curving around the leg regions by at least about 150 degrees.

44. A liquid absorbent article as defined in claim 43, wherein the leg areas comprise from about 35% to about 85% by weight of the absorbent structure.

45. A liquid absorbent article as defined in claim 43, wherein the leg areas comprise at least about 40% by weight of the absorbent structure.

46. A liquid absorbent article comprising:
an outer cover and an inner liner, the article including a front region and a back region, the article further including leg regions, and wherein, when the article is positioned about a user, the leg regions extend a determined distance downwardly along the legs of a user; and
an absorbent structure positioned in between the outer cover and the inner liner, the absorbent structure comprising a crotch area positioned between the front region and the back region of the article, the crotch area being in communication with a pair of downwardly extending leg areas, the leg areas being positioned within the leg regions of the article and configured to lay adjacent to the inner thighs of a user, the leg areas having sufficient liquid holding capacity to hold at least about 30% of expected liquid loading.

47. A liquid absorbent article as defined in claim 46, wherein the liquid absorbent article has a liquid holding capacity of at least about 200 grams without becoming saturated.

48. A liquid absorbent article as defined in claim 46, wherein the liquid absorbent article has a liquid holding capacity of at least about 1000 grams without becoming saturated.

49. A liquid absorbent article as defined in claim 46, wherein the article comprises a feminine hygiene product and wherein the absorbent article has a total liquid holding capacity sufficient to hold from about 7 grams to about 50 grams of menstrual fluid without becoming saturated.
